(19) United States
(12) Patent Application Publication
(10) Pub. No.: US 2016/0038692 A1
(43) Pub. Date: Feb. 11, 2016

(54) Internally controlled cell spray dispenser

(71) Applicant: Andrej Bahoric, Whitby (CA)

(72) Inventor: Andrej Bahoric, Whitby (CA)

(21) Appl. No.: 14/121,144

(22) Filed: Aug. 6, 2014

(51) Int. Cl. A61M 11/02

(52) U.S. Cl. CPC A61M 11/02; A61M 2202/09; A61M 2210/04

(57) ABSTRACT

Internally controlled cell spray dispenser comprises embodiments that are being used to generate spray by controlling a contact between solutions of different cellular composition and the incoming compressed air produced and triggered by an external source of compressed air. In order to obtain desired quality of spray the present apparatus uses the compressed air regulator, and elastic properties of the air channel valve to control both the circumference of the air channel, and the flow of air in to the frontal cone where the main contact between the two fluids is taking place. Reduced air channel is causing a reduction in flow of air and an increased air pressure in the air channel and in the frontal cone promoting discharge of thicker liquids and larger tissue grafts. Releasing the pressure exerted on the air channel valve is causing the air channel to elastically rebound allowing more air under less pressure to enter the frontal cone promoting a discharge of smaller particles suspended in thinner media.

This application claims a benefit of the Provisional Patent Application Number U.S. 61/958,951; entitled: Internally Controlled Multipurpose Spray Dispenser. Filed Aug. 12, 2013. U.S. Classification 222/4,251,195,153,173,226,211,251,526,630,272. 239/1,8,346,74,310,142,302,143,311,337,341,366,368,369,401,569. 251/4,7,8,9,84,85,149.7,160,341. 366/101,150.1,162.4,208,221. 261/19,30,38,44.1,55,64.3,71,74,75,87.108,119.1,121.4. Patent search: U.S. Pat. No. 7,641,898 B2, U.S. Pat. No. 5,284,299 A, Filled: Aug. 2, 2014.

### FIELD OF INVENTION

The present invention relates to an apparatus and a method for controlled spray dispensing of living cells entrained in liquid media to surfaces.

### BACKGROUND OF INVENTION

Surgical closure of large wounds is regularly done by covering them with partial or full thickness skin grafts removed from the healthy site of the same patient. However, when there is not enough skin, viable cells derived from the fragmented and enzymatically de-aggregated patient's own skin are being used to resurface wounds. Separated cells can be expanded in culture, or sprayed over the wound surface. Cultured epidermal cells after 14 days of incubation are giving rise to a fragile epithelial sheets suitable for covering smaller lesions.

Spray application of liquid media with entrained cells, proved to be a faster and more practical way for resurfacing wounds and for covering constructs in medical engineering.

A number of devices presently used for this purpose are primarily designed for spraying medical adhesives. However, in the absence of better means for dispersing cells, these devices are loaded with cellular material. Assembled of a single or a plurality of syringes these devices are actuated by manually engaged plungers in order to push the content in each syringe in to the path of the compressed air entering devices at their discharge orifices.

A Baxter's Tisseel system the Epicel sprayer as well as a Spray Gun (Pittsburgh) are working on the same principle.

A short-coming of these sprayers is a tendency of cells to settle at the bottom of loaded syringes giving an un-even distribution of sprayed cells. There is also a warning issued by the FDA about the possibility of inducing an air embolism by sprayers whose compressed air is discharged to close to the open blood vessels in the wound bed.

Most of the sprayers currently involved in spraying cells have small and rigid orifice which is also being used to regulate spray. However, rigidity and the small size of these orifices are limiting discharge from these sprayers to watery solutions and individual cells. Obtaining single cells from skin grafts is a long process which can be accelerated with a use of high concentration of trypsin as it is in the case of the Re-Cell system. However, using concentrated tissue enzymes in a tissue dissolution process is rising a possibility of finding their residues in the spray solution prompting FDA concerns.

A compressed air driven multi-channel sprayer of the TiCell System (Application No. US 20050026275 A1-abandoned) is satisfying operating room timings, while minimizing the use of enzymes in a tissue dissolution process by its ability to spray cells obtained at various stages of the tissue dissolution process. However, due to high sensitivity of the particular system to alterations in air pressure, it is difficult to obtain an uniform spray when the air pressure is changing. Lowering the external air pressure is giving poor quality of spray, while an increased external pressure is causing a bounce back effect of cellular material from the sprayed surfaces.

Cells can be also dispersed by using an air-jet suction effect as described in U.S. Pat. No. 7,641,898 B2.

It is clear that there is a great need in the art for an improved method and system for dispensing cellular material.

### SUMMARY OF INVENTION

The present invention relates to an apparatus and a method designed to generate and to apply a spray, comprising prepared
de-aggregated cellular material in variety of configurations entrained in liquid media of variable consistency. The present apparatus has a capability of discharging solutions containing, single cells and cell aggregates obtained by mechanical and enzymatic dissolution of skin grafts as wall as a discharging of miniature tissue grafts mechanically separated from the harvested tissue stored in graded supply receptacle. Discharging process of the cellular material is initiated by an influx of externally generated compressed air which under a preset pressure enters the apparatus, interacting with a liquid content within the apparatus causing agitation, saturation and discharge of the content. Controlling mechanism within the said spray apparatus assures an uniform discharge of the content regardless of the composition of the sprayed solution.

Conduits in direct contact with fluids and a fragile content of the dispenser are open, smooth and made of bio-compatible material.

### BRIEF DESCRIPTION OF DRAWINGS

FIG. 1 Is a schematic representation of the cross-section of the said dispenser. It is shoving embodiments as they are positioned within a dispenser.

FIG. 2 Shows an arrangement of flexible embodiments of the dispenser.

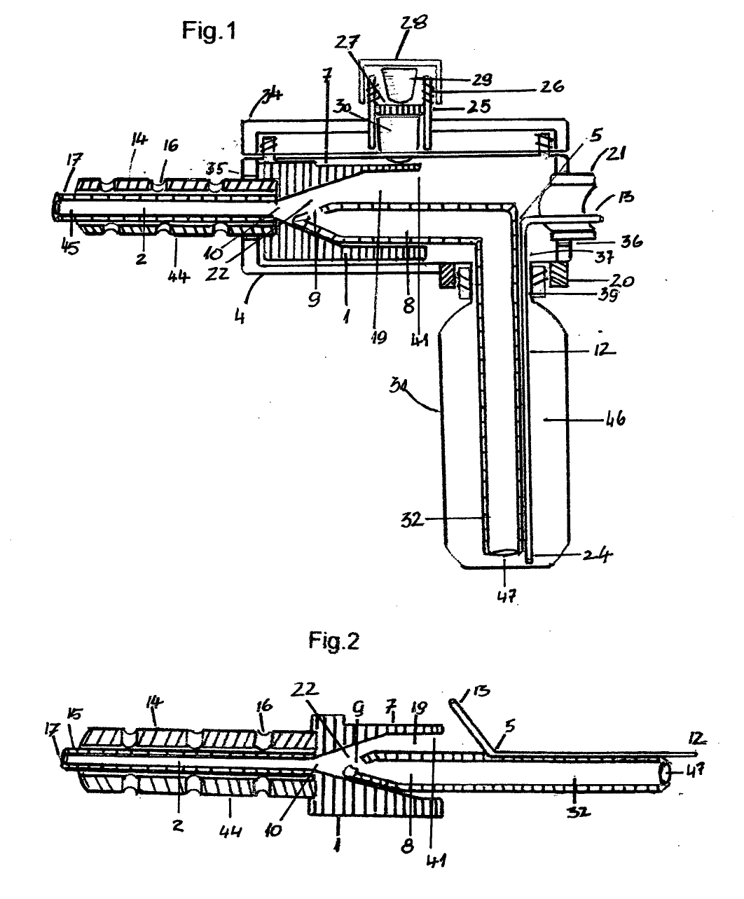

### DETAILED DESCRIPTION OF EMBODIMENTS OF THE PRESENT INVENTION

Referring to the figures in the accompanying drawings, the illustrative embodiments of the present invention will now be described in great technical detail, wherein like parts are indicated by like reference numbers.

The present invention having embodiments retained in a manner that will ensure their position and function, comprising a casing (34,4) a receptacle made of hard plastic material preferably polyurethane, having a plurality of conduits (1,2,32,8,12) all made of flexible plastic material preferably medical grade silicone.

Also included is a supply receptacle (31) a holder for storing the dispensing material preferably made of hard plastic material or pyrex glass. Embodiments included in the dispenser are assembled together with a use of bio-compatible adhesives such as epoxy and medical grade silicone glue. A casing (4,34) a vertically and radially extended receptacle constructed of two separate look like sections with both sections permanently closed on one side. The wall of the lover section a body (4) of the casing, has threads on its open end that correspond to threads on the inside surface of the open end of the basically like cover (34).

In order to form a complete casing, a removable closure between the body and the cover is secured by twisting and bonding these two components together.

The frontal aspect of the body of the casing has an opening (35) for passing the outwardly extended cell spray nozzle (44). A posterior wall of the body of the casing is having an opening (36) with an outwardly directed adapter (21) of a compressed air inlet (36) secured in the said opening.

A said adapter has a mean for attaching the plastic hose for receiving compressed air from an external compressed air actuator. An opening (37) on the posterior end of the base of the casing is fitted with a hollow and outwardly directed plastic adapter (20), having inside threads that correspond to outside threads on the free end of the supply receptacle.

A gas-tight detachable closure between these two members form a central channel (39), communicating between the cavity of the casing and a cavity of the receptacle. The said channel accommodates both the liquid conducting tube (32) and the air bubble tube (12,5,13). According to an aspect of invention, sealed in to a center and extended on both sides of the surface of the of the cover (34) of the casing is a hard plastic tube (25) of the compressed air regulator (26). A cavity of the said tube contains a compression mechanism of the regulator. The external wall of the upper portion of the plastic tube has outside threads (35), complementary to threads on the inside wall of the twisting cap (28), movably fitted on this side of the tube.

Rotating cap is adapted to operatively engage the compression mechanism within the plastic tube. The said mechanism which as a follower derives its motions following the motions of the cap, comprises an elastic membrane (27) stretched and retained over the rigid plastic retainer in the middle portion of the lumen of the plastic tube, and two hard plastic inserts inserted in to the plastic tube on both sides of the said elastic membrane.

The upper insert (29) having seat on a surface of the elastic membrane protruding on the opposite side beyond the upper edges of the plastic tube, where is becoming engaged by the inner surface of the plastic cap.

A lower insert (30) which is interposed between the said elastic membrane and the rear portion attenuated dorsal wall (7) of the central chamber (1), having a seat on the very end of the said attenuated dorsal wall (7) is being a part of the air channel valve (41) located below the said wall.

The apparatus of the present invention is having a plurality of flexible tubular conduits comprising: a central chamber (1) a cell spray nozzle (44) and the liquid conducting tube (8,32,47) with the bubbler tube (5,12,13) incorporated in to back wall of the said liquid conducting tube.

A central chamber (1) is a wide thick walled axially extended elastic cylinder located within the cavity of the plastic casing, between the frontal wall of the said casing and the opening for the passage of the liquid conducting tube on the posterior end of the base of the plastic casing (37).

A said chamber (1) comprises a cavity with a smaller frontal and a larger rear annular opening.

It also comprises, an attenuated dorsal wall (7) having a collapsible posterior portion, and the frontal cone (22) a cone shaped frontal portion of the cavity of the central chamber outlined frontally by the entrance in to the cell spray nozzle (44), and on the rear by the sloped terminal portion of the liquid conducting tube (8).

A Central chamber also comprises an air channel (19), a space defined by the inner surface of the said attenuated wall (7) and the upper wall of the terminal portion of the liquid conducting tube (8), wherein sealed in to the floor of the rear portion of the cavity of the central chamber. The air channel having preferably 0.192″ID connects the cavity of the said casing and the cavity of the frontal cone (22) of the central chamber.

A said chamber is having an air channel valve (41) comprising a rear terminal portion of the attenuated dorsal wall of the central chamber, defining an annular entrance in to the air channel (19), the outside wall of the terminal portion of the liquid conducting tube and a lower insert of the compressed air regulator (26) having a seat on the end portion of the attenuated dorsal wall of the central chamber.

The central chamber is kept in the position within the plastic casing by an out-flow cell spray nozzle (44), which is coupled to the frontal opening on the wall of the central chamber and extended outwardly tightly fitted through an opening on the frontal wall (35) of the plastic casing, and by the terminal portion of the liquid conducting tube (8) sealed in to the cavity of the central chamber.

A cell spray nozzle comprises a small bore thin walled tube (2) having preferred sizes of 0.057″I.D.×0.060″O.D, 0.062″I.D×0.095″O.D. extended within a wider thick walled tube (14) made of the same material having a preferred size of ⅛″I.D.×¼″O.D. A wall of the smaller tube (2) is expanded and sealed to the inside wall of the wide tube (14) at the point of the coupling of the said nozzle to the annular opening of the cavity of the central chamber.

Both tubes are also sealed at the terminal end of the said wide tube enclosing a space between these two tubes. A discharge end of the said smaller tube is protruding slightly (45) beyond the external limits of the wide tube ending with a single discharge orifice (17). There is no deflector or discharge modification means placed in or on the cell spray nozzle. As an embodiment a surface of the wider tube has a series of openings-vents (16), allowing expansion of the inner tube when the solution is being discharged.

A present invention comprise a liquid conducting tube (8,32,47) a thick walled wide lumen tube with inside diameters ranging from 0.104 to ⅛ inches. Terminal portion of the said tube (8) is sealed to the base of the cavity of the central chamber ending close to the frontal cone (22).

The very end of the terminal portion of the said tube is compressed and sealed assuming a sloped shape. An opening (9) facing forward and upward is made on the sloped portion of the said tube. There is a space formed between the outside wall of the said tube and the inner surface of the attenuated wall of the central chamber defining the air channel.

A said liquid conducting tube outside of the central chamber (1) but still within the casing the liquid conducting tube (8), assumes an elbow shaped curve, which straightens up when the said tube enters the central channel (37) on the base of the plastic casing. Upon entering the supply receptacle (31), the said tube is lying freely within the said receptacle ending about 5 mm above the bottom of the receptacle (47).

The air bubbler tube (5,12,13) according to one aspect of invention, is an open ended silicone rubber tube having an inside diameters between 0.030 and 0.040 inches incorporated in to the back wall of the liquid conducting tube (32). A portion of the said tube within the supply receptacle protrudes beyond (24) the entrance in to the opening of the liquid conducting tube ending very close to the cone shaped bottom of the supply receptacle.

In the opposite direction the said tube progresses in to a space within the plastic casing still attached to the liquid conducting tube. Upon entering the casing it separates (5) from the said tube ending secured within the adapter (21) of the compressed air inlet. It protrudes (13) slightly beyond the entrance in to the said adapter. The present invention uses supply receptacles (31) made of glass or hard plastics heaving a cone shaped or rounded bottom.

The supply receptacle is kept in position by a detachable closure made between the outwardly protruding adapter (20) on the plastic casing and the corresponding fitting on the neck of the receptacle. A spray dispensing of the cellular material entrained in liquid media and stored in the supply receptacle is done, by an influx of compressed air switched on at the external source of compressed air.

A said air enters the apparatus under constant pressure between 40-60 PSI by way of a plastic hose fitted in a detachable closure to the plastic adapter of the compressed air inlet (21). A small portion of said compressed air enters the supply receptacle by way of the bubbler tube (5,12,13) before entering the cavity of the plastic casing wherein the compressed air rebounds from the curved bottom of the receptacle producing an stream of upward directed air bubbles.

A said bubbles agitate and saturate with air the liquid content in the said receptacle, without causing discharge of the cellular content. A main stream of compressed air entering the cavity of the plastic casing, is being diverted in to the supply receptacle by way of the central channel (39) where it forces an already agitated and air saturated cellular content in to the flow path of the liquid conducting tube (47,32,8) and in to the frontal cone (22) of the central chamber, by way of an open air channel.

Both fluids are intermingling in the frontals cone of the central chamber channel (19) producing an air saturated and pressurized mixture of fluids and dispersed cells, wherein being discharged to the fluid path of the small elastic tube through an expanded entrance (10) in of the cell spray nozzle (44) and in to the environment through an open discharge orifice (15) on the protruding portion (17) of the small tube.

Elasticity of the small tube and the present vents (16) on the large outside tube (44) is facilitating the expansion of the small tube allowing larger particles to proceed un-impeded toward the discharge orifice of the nozzle. Adequate spray can be produced by the present apparatus by controlling interaction between cell containing liquids and the compressed air within the frontal cone of the central chamber.

Flow of air in to the frontal cone is regulated according to the size of the cellular content and the consistency of the spraying solution to be discharged by using air channel valve actuated by the compressed air regulator (26).

The compressed air regulator (26) is acting on the air channel valve (41) that controls the circumference of the air channel (19) and the flow of air through the said channel.

A downward twisting the regulator's cap (28) from the stand by position, presses the upper insert (29) of the said regulator, engaged by the said cap pressing the elastic membrane (27) below the said insert, causing an expansion of the said membrane and the compression of the lover cone (30) of the regulator. Compressed lover cone presses on the air channel valve (41) reducing the circumference of the air channel (19).

Reduced circumference of the air channel causes decreased flow but increased air pressure in the frontal cone (22) of the central chamber promoting discharge of thicker liquids and larger tissue grafts. Movements of the cap in opposite direction toward the stand by position, releases the pressure on the air channel valve which rebounds due to the elastic properties of the attenuated dorsal wall (7) allowing more air under less pressure to enter the frontal cone promoting discharge of smaller particles suspended in thinner media.

While I have shown and described what I regard to be the preferred embodiments of my invention it will be obvious to those skilled in the art that modifications may be made without departing from the invention in its broader aspect.

1. An internally controlled cell spray dispenser, having embodiments capable of dispensing viable cells while operating from source of compressed air, comprising
a casing having a compressed air regulator inserted in to the said casing and a supply receptacle attached to the base of the casing, all made of hard plastics preferably polyurethane,
a central chamber having a spray nozzle and the liquid conducting tube with a bubbler tube incorporated in the wall of the said tube all preferably made of medical grade silicone rubber.

2. An internally controlled cell spray dispenser of claim 1, having a casing, wherein comprising
a cavity communicatively connected with a supply receptacle and with a source of compressed air, having
an upper flat surface having an opening for the tube of the compressed air regulator and a lover flat surface, having means for attaching the supply receptacle, having
a first and second side facing opposite directions, comprising an opening for the passage of the cell spray nozzle and an opening for the compressed air inlet on the opposite side of the said casing.

3. A casing of claim 2 having a cavity within said casing communicatively connected to the supply receptacle and to the source of the compressed air, comprises
a compressed air regulator pivotally inserted and extended on both sides of a said upper surface of the said casing, comprising
a hard plastic tube and a cap rotatably engaged to threads on the external free end of the said tube, having
a movable followers within the said tube, having two hard inserts separated in the middle of the said tube by an elastic membrane, having
an upper insert wherein engaged by a said membrane and a said cap of the said regulator, also having
a lover insert of the said regulator engaged to a lover surface of a said elastic membrane, having seat on the free end of the attenuated dorsal wall of the central chamber, wherein
a said casing of claim 2 is, further comprising
a supply receptacle detachably connected to the outlet on the base of the plastic casing, wherein having
a rounded or cone shaped bottom in close proximity to the extended portion of the air bubbler tube, having further
a communication with a central chamber through the liquid conducting tube and with the cavity of the plastic casing through the open central channel.

4. An internally controlled cell spray dispenser of claim 1, having embodiments capable of dispensing viable cells while operating from source of compressed air, comprising a casing of having a cavity within said casing communicatively connected to the supply receptacle and the source of the compressed air having a tube of the compressed air regulator inserted in to the cover of the said casing, further comprising
a central chamber a cylindrical structure having a cavity within the said chamber having fontal and rear opening, wherein
a said chamber being secured within the cavity of the plastic casing by the cell spray nozzle, coupled to the frontal opening of the said chamber and by the liquid conducting tube sealed to the floor of the inside surface of the said chamber, further comprises
an attenuated dorsal wall having a resiliently yieldable posterior free portion being a seat for the lover insert of the compressed air regulator, wherein said chamber comprises
an air channel, a tubular structure having preferred 0.192″I.D. located between the inside surface of the said attenuated wall and the outside wall of the terminal portion of the liquid conducting tube sealed to the inside surface of the cavity of the said chamber, connecting the frontal cone and the cavity of the plastic casing, comprising
an air channel valve comprising a posterior free end of the attenuated dorsal wall of the central chamber, the air channel, and the outside wall of the liquid conducting tube just below the posterior free end of the said attenuated wall and the lover insert of the compressed air regulator, having seat on the surface of the said wall, wherein a said chamber further comprises
a frontal cone an axially extended chamber like element along the egress rote, outlined by the cell spray nozzle coupled in to the frontal opening of the said cone and by the terminal portion of the liquid conducting tube, connecting a said frontal cone with a supply receptacle,

5. A said central chamber of claim 4, wherein secured within the cavity of the plastic casing, having a cavity within a said chamber having a frontal and rear annular opening and an attenuated dorsal wall, and a frontal cone, having an air channel and air channel valve, further comprising
a cell spray nozzle coupled to the frontal opening of the central chamber, comprising
an inside small bore thin walled tube with an optional size of 0.057″I.D.×0.060″O.D, or 0.062″I.D.×0.095″O.D., extended inside a wide thick walled tube heaving a preferred size of ⅛″I.D.×¼″O.D.,
a said thick walled tube having plurality of openings extended through the wall of a said tube, having
a small inside tube extended beyond the terminal portion of the wide tube ending in a discharge port of the nozzle, having
a said tubes bonded together at junction with a frontal cone and at their external terminal portion, having
a gap between the outer surface of the inner tube and the inner surface of the outer tube, forming a space between tubes.

6. A said central chamber of claim 4, wherein positioned and secured within the cavity of the plastic casing, having a cavity within a said chamber having a frontal and rear annular opening and an attenuated dorsal wall and a frontal cone with frontally attached a cell spray nozzle and an air channel having an air channel valve, also comprises
a liquid conducting tube having, an optional size of 104″I.D.×192″O.D. or ¼″I.D.×⅛″O.D., having
a frontal portion of the said tube sealed in to the base of the cavity of the central chamber, wherein
a said tube ending at the entrance in to the frontal cone with a sloped surface, having an open upward and frontally directed orifice with, having
a distal portion of the said tube laying free within the cavity of the supply receptacle.

7. A liquid conducting tube of claim 6, having a sloped terminal frontal portion bearing an upward and forward directed opening sealed in to the base of the cavity of the central chamber ending at the entrance in to the frontal cone, wherein distal portion of the said tube is laying free within the cavity of the supply receptacle, comprises
an air bubbler tube incorporated in to the posterior wall of the said liquid conducting tube, heaving
a proximal segment secured within the compressed air inlet of the plastic casing, having
a distal portion of the said tube protruding about 2 mm beyond the open end of the liquid conducting tube, having
a distal portion of the said tube ending 1 mm above the rounded or cone shaped bottom of the supply receptacle.

9. An internally controlled cell spray dispenser of claim 1, wherein generates spray by interacting a pre-formed solution comprising single cells cell aggregates and miniature tissue grafts entrained in liquid media and stored in the graded supply receptacle, with an externally generated compressed air, having constant pre-set pressure between 40-60 PSI triggered at the source of the compressed air, wherein embodiments comprised in the dispenser are being used to regulate this interaction, wherein
a said compressed air entering a supply receptacle through the air bubbler tube protruding the compressed air inlet, before the main body of the compressed air enters the casing, wherein
a said compressed air rebounding from the cone shaped bottom of the said receptacle agitates and mixes with air a content in the receptacle without discharging a said content, wherein
a said agitated content is being discharged from a said receptacle by a positive pressure of the main body of air entering casing through a said inlet and a receptacle through the central channel, wherein
a said discharged content enters a flow path of liquid conducting tube and in to the frontal cone, wherein
a said content in the frontal cone is being exposed to compressed air entering a said cone from a said casing by way of the open air channel, wherein
a said air generates spray by mixing, pressurizing and saturating with air a content in a frontal cone, wherein
a pressurized mixture of air and liquid, in the frontal cone is being discharged in to the cell spray nozzle and in to the environment, wherein
a said process is being controlled and modified by a manually actuated compressed air regulator and the elastic properties of the air channel valve, wherein
a downward rotation of the cap of the said regulator is engaging followers within the organization increasing pressure on the lover cone of the regulator, and on the attenuated wall of the central chamber actuating the air channel valve, wherein
a compressed resiliently yieldable wall of the air channel is holding it against its sprig bias, reducing a flow of said air in a said channel, wherein
a decreasing flow of air in the air channel wherein increasing a pressure of air in the said frontal cone wherein promoting spray discharge of larger size cellular material entrained in thicker media, wherein
a rotation of the cap in opposite direction from the compressed position de-actuates the air channel valve, causing the air channel to elastically rebound from the compressed position, wherein promoting discharge of smaller cells suspended in thinner media, wherein
a regulation of a spray can be induced by manually changing the circumference of the air channel according to the composition and the consistency of the cell containing solution.
a total closure of a said air channel makes the dispenser un-operable.
